(19) United States
(12) Patent Application Publication
(10) Pub. No.: US 2019/0365607 A1
(43) Pub. Date: Dec. 5, 2019

(54) Biometric Pill Dispensary System

(71) Applicants: Michael R. Kugler, Bridgeville, PA (US); Jeffrey M. Sgro, Bridgeville, PA (US); Ashumi P. Rokadia, Bridgeville, PA (US); Christopher V. Rodi, Bridgeville, PA (US); Joshua D. Milteer, Bridgeville, PA (US); Matthew S. Detar, Bridgeville, PA (US); Suraj S. Bokil, McDonald, PA (US)

(72) Inventors: Michael R. Kugler, Bridgeville, PA (US); Jeffrey M. Sgro, Bridgeville, PA (US); Ashumi P. Rokadia, Bridgeville, PA (US); Christopher V. Rodi, Bridgeville, PA (US); Joshua D. Milteer, Bridgeville, PA (US); Matthew S. Detar, Bridgeville, PA (US); Suraj S. Bokil, McDonald, PA (US)

(21) Appl. No.: 16/000,533

(22) Filed: Jun. 5, 2018

(51) Int. Cl. A61J 7/04; G16H 20/13; A61J 1/03; A61J 7/00

(52) U.S. Cl. CPC A61J 7/0445; G16H 20/13; A61J 7/0436; A61J 1/035; A61J 7/0076; A61J 7/0481

(57) ABSTRACT

A pill dispenser intended to prevent overmedicating and overdosing on addictive medication. A blister pack is first loaded into the device and then locked within by the pharmacist. The device is then programmed by the pharmacist according to the prescribing physician's prescription. The device will only dispense the appropriate amount of medication to the patient at a designated time interval when the correct fingerprint is scanned. The patient cannot access his or her future dosages until the correct dosing time has elapsed, nor can any other person access the medication within the device. If the fingerprint is that of the patient, a stepper motor can be used to turn the blister pack inside, allowing the striker mechanism to dispense the user's medication. If the fingerprint is not recognized, the device will not respond, and the person will not have access to the drugs enclosed within Likewise, if the user tries to use the device before the correct dosing schedule, it will not respond, thus preventing overmedication. Overall, the device uses integrated technologies to create a secure dispensary system for the highly addictive medications held within.

### CROSS REFERENCE TO RELATED APPLICATIONS

The applicant hereby claims benefit of U.S. Provisional Patent Application No. 61/515,159 filed Jun. 5, 2017. The specification and drawings of said provisional patent application are incorporated herein by this reference as though set forth in full.

### BACKGROUND OF THE INVENTION

The administering of prescription medication has come to be a problem in the healthcare industry. Most medicines are taken orally in the form of tablets and capsules. Traditionally, these tablets are stored in a bottle that can be subject to tampering. This presents the opportunity for the overmedication, either by accident or deliberately, of highly addictive painkillers and can ultimately lead to drug dependency and death in the long-term from an overdose. Apart from the housed drug being abused, it also allows for non-intended individuals to access the medication. Situations such as these occur to a staggering degree, resulting in overdoses and deaths.

Housed within the standard orange pill bottle, there is no secure means of protecting the drug apart from a child-proof locking mechanism. Apart from adults being able to bypass this lock, it is also very easy for a child to obtain a lethal dose of medication held within the bottle. When a patient obtains a prescription from a doctor, it becomes his or her responsibility as to whether or not he or she follows the dosing instructions. Furthermore, in many cases, friends or acquaintances of a person who has been prescribed such medication will attempt to steal pills in order to soothe undiagnosed pain he or she is experiencing. In the specific case of opioids, about fifty-five percent of users who received prescription painkillers obtained them from a friend or relative, not a medical professional. Even when these drugs are prescribed by a doctor, they are often in large quantities, leaving the patient with an excess or an obligation to overmedicate beyond his or her needs. In either instance, this increases the possibility for abuse of the prescribed medication by the intended recipient or another person.

Lawmakers have been struggling to find a solution to the opioid crisis aside from rehabilitation efforts. Rehabilitation efforts in recent years have demanded billions of dollars from the federal government, in which only a small percentage of people found these efforts to be beneficial and actually treat his or her addiction. Children are being stripped of their futures by accidentally obtaining a family member's medication and beginning the road of addiction.

The main problem with standard pill bottles is that anybody can access its contents as there is no protective lock or seal. Therefore a need exists for a secure pill dispensary system for the highly addictive medications held within that can help curb the staggering statistics of drug addiction and drug dependency.

### SUMMARY OF THE INVENTION

This invention relates to a pill dispenser housing prescription medication. A secure device can be loaded and locked with a blister pack of medication at the pharmacy. This will allow only one pill to be dispensed at a prescribed interval designated by the physician. An integrated fingerprint scanner can be used to prevent other people from unlocking the device and accessing the medication.

The current device disclosed herein is designed to mitigate the abuse of drugs by locking down the medication housed inside for its intended user. The currently disclosed device eliminates the problem with standard pill bottles, that anybody can access their contents as there is no protective lock or seal, because it can be configured to be unique to only one individual. The device disclosed herein can be assembled by a pharmacist and the medication can be inserted in a custom blister packaging made to fit the device. It is then programmed for the specific patient with the correct prescription and dosage information. With this, it utilizes an internal timer to prevent obtaining too many pills at once.

With this invention, a greater responsibility in the handling of prescription drugs in instituted between the patient and prescriber. Users are forced to strictly follow the guidelines of the physician. This also creates a greater sense of accountability that the housed medication is not abused. In addition, should the user not require all of their medication, the unused pills are returned in the blister pack to the pharmacy upon return of the device.

Overall, the device allows the user to precisely dispense their medication safely and effectively. With enhanced security measures, including without limitation biometrics, locking systems, and rigid materials, the device is a safer way to store and dispense prescription medications.

### BRIEF DESCRIPTION OF THE DRAWING(S)

Many aspects of the device and its components may become clearer with reference to the figures and drawings. The components are not necessarily drawn to scale, but illustrate the functioning components.

FIG. 1 illustrates a front isometric view of the device as completely assembled and ready for use.

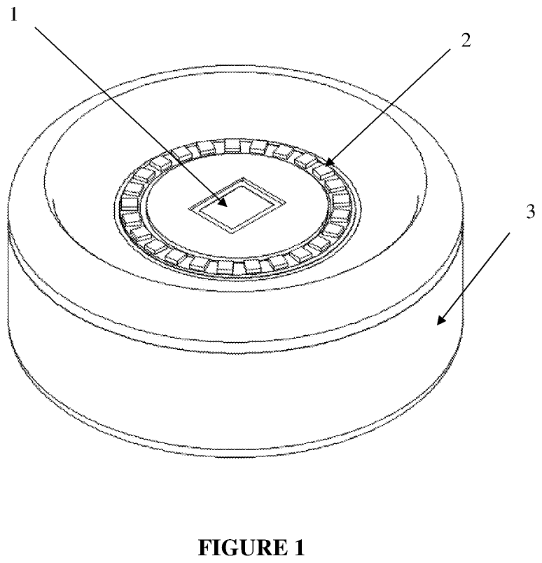

FIG. 2 illustrates an exploded view of the main components of the device with the top portion of the outer shell cutaway to review the electronics and motors mounted inside the outer shell and the lid removed but with the blister pack shown both by itself and as positioned within the outer shell.

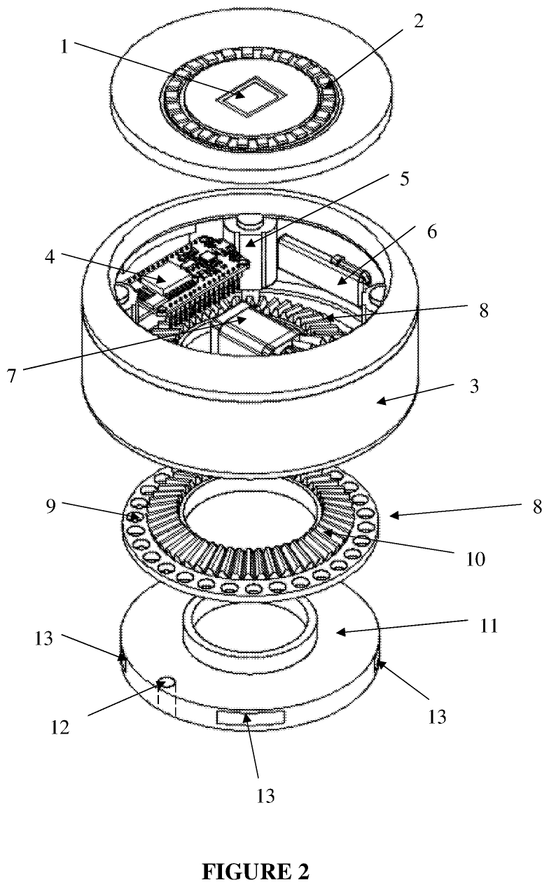

FIG. 3 illustrates an internal view of the device with the top portion of the outer shell removed to show the interior.

FIG. 4 illustrates the lid with the blister pack positioned on top of the lid as assembled.

FIG. 5 illustrates the blister pack, which will rotate within the device when turned by the pinion gear on the stepper motor.

FIG. 6 illustrates the striker assembly, which will be used to punch out the pill once the stepper motor turns the blister pack.

FIG. 7 illustrates a diagram of the wiring used within the device to allow it to function.

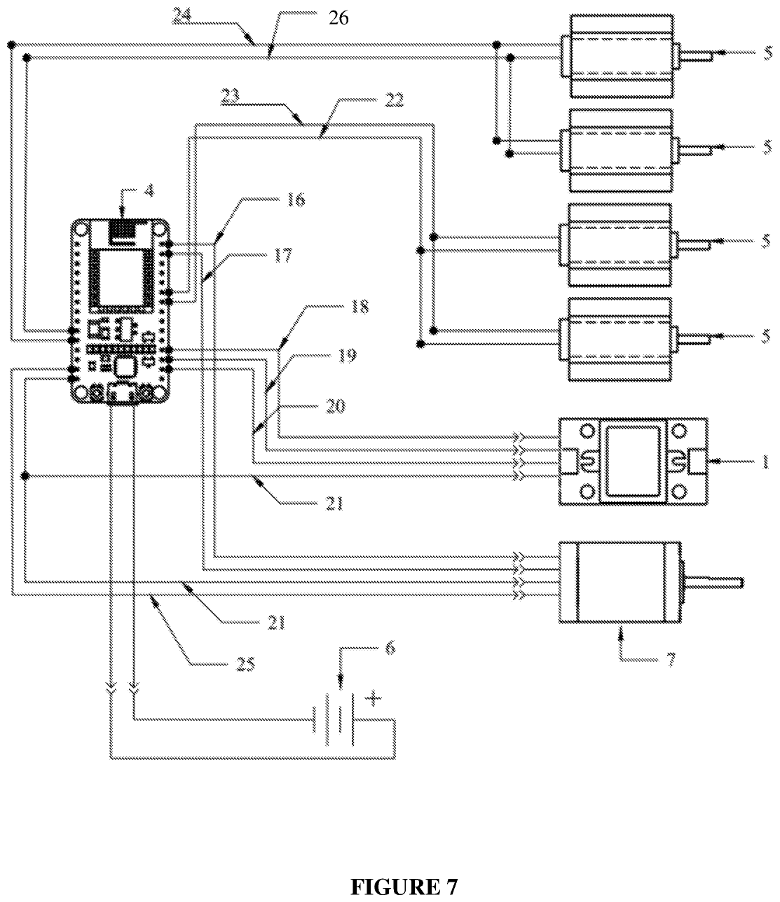

FIG. 8 illustrates a flow-chart diagram of the feedback system which the fingerprint sensor goes through to analyze the finger of the patient.

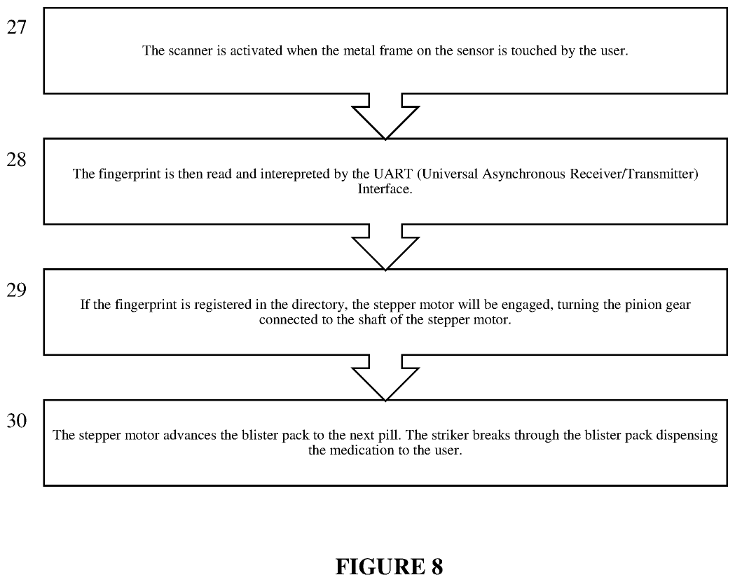

### DESCRIPTION OF THE INVENTION

The device and its function ensure a safe dispensary system using multiple technologies. The following description details the apparatuses contained within, in accordance with the figures. They offer clarification to the specific features and design considerations of the device.

The device comprises a two-piece system comprising an outer shell 3 and a lid 11 to simplify the loading process at the pharmacy. Both pieces can be assembled together by a pharmacist before being distributed to the patient. In one preferred embodiment, both pieces can be created out of Polylactic Acid (PLA) plastic, a 3D printed material. PLA has a tensile strength of 8,840 PSI to 9,500 PSI and a flexural strength of 6,950 PSI to 16,000 PSI. The plastic can also withstand high temperatures ranging between 315° F. to 388° F. The combination of strength and heat resistance allows the device to be exposed to harsh conditions through daily patient use.

The main purpose of the parts of the housing 3 and 11 being made of plastic is to ensure wireless communication between the device and the pharmacy. Since the device is reusable, it has to be able to easily sync with a pharmacist's computer to program a new user's prescription and fingerprint. By utilizing wireless technologies, the device also has a less likely chance of being tampered with as there are no external ports. All communication takes place on a wireless docking station located at the pharmacy. Despite there being other materials perhaps stronger than PLA, nothing is completely tamper-proof. A determined addict can and will break into anything. The purpose of this device is not to cure drug dependency, but prevent one from happening in the first place.

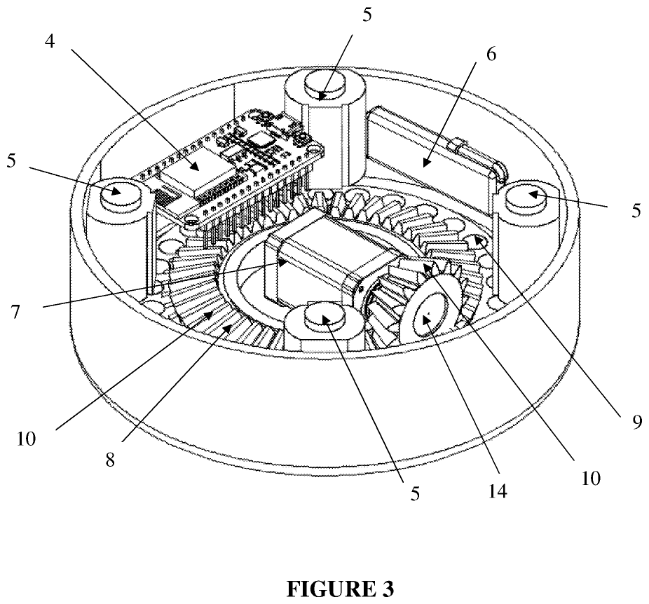

In reference to FIG. 3, the outer shell 3 is the central component of the two-piece system, which houses the device's main electronics including the microprocessor 4 and battery 6, motors 5, and blister pack 8 as well as the medication 9 within the blister pack. FIG. 3 depicts the outer shell 3 with the top portion thereof to which the sensor 1 is mounted removed or cutaway in order to see the components as mounted with the outer shell 3. The outer shell 3 acts as the main barrier between patient and the medication 9. The outer shell 3 of the device is ergonomically designed to be portable for the patient. Its compact size, albeit a bit larger, is comparable to the compact size of the traditional pill bottle, fitting within a purse or even inside of a pocket. On the lid are four female notches 13 corresponding to the four servo motors 5 mounted within the outer shell 3 which combined with bars affixed to the servo motors 5 function to lock the outer shell 3 and lid 11 pieces together. When the pharmacist locks the device, he or she will place his or her finger on the fingerprint scanner 1, turning the servos and locking the two pieces together.

The internal locking mechanism can be broken down into two parts. The outer shell 3 houses four servo motors 5, which are mounted on the interior of the outer shell 3. The motors 5 have small, one-inch bars affixed to them that allow them to lock to the lid 11 when rotated. When the pharmacist places his or her finger on the device, it will prompt the four motors to rotate 90 degrees, securing them into the four female notches 13 on the lid 11. The main purpose of having a removable piece is to easily load the blister pack 8, while keeping its contents secure. Likewise, once the device is returned to the pharmacist, he or she will place his or her finger on the biometric scanner 1 allowing the four servo motors 5 to rotate back to their original positions and unlock the device. At this point the pharmacist can remove the lid 11, change out the blister pack 8, and reprogram the device for a new user.

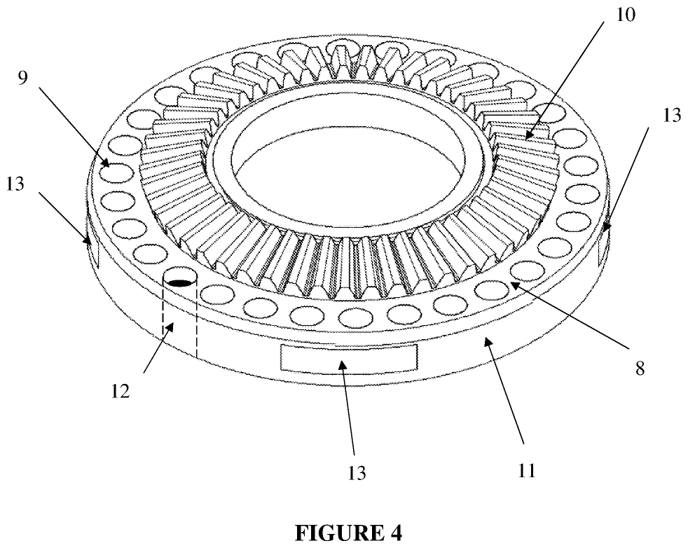

The blister pack 8 rotating assembly (FIG. 4) of the device is the main means of distributing the medication 9 to the user. It contains the blister pack 8, which is loaded by the pharmacist prior to locking the device. This lid 11 is made of the same PLA plastic used on the outer shell, making it strong and resilient against tampering. Its circular design fits into the bottom of the outer shell 3 and then locks into the notches 13 provided to hold firmly inside of the device. The blister pack 8 then rests on the lid 11 to allow for the rotation of the internal blister pack to dispense the medication outward. On one end of the lid, there is a hole 12 where the medication is outwardly dispensed to the patient. The outer edge of the lid 11 fits tightly against the bottom of the outer shell 3, protecting the security of the device from being compromised. Pharmacists are the only ones able to remove this lid 11, upon the device being returned or it being redistributed.

The medication is dispensed from the device through a striker pin 15 mounted to the interior of the outer shell 3. This pin 15 is comprised of the striking tip and the spring. When the blister pack 8 rotates, the pin 15 slides upward and compresses the spring, thus storing potential energy. Upon the next pill 9 being ready, the blister pack 8 rotates and aligns with the pin 15. This potential energy is released and the spring expands, allowing it to punch downward, breaking the blister pack 8 and dispensing the medication 9. Only when the next pill 9, pin 15, and hole 12 in bottom lid 11 are aligned shall medication be dispensed. The main purpose of the striker 15 is to precisely dispense the medication 9 from the blister pack 8 into the user's hands. Since the mechanism is fastened inside, it remains secure from damage. The pin 15 requires no electrical input as it is a simple machine containing merely a spring and striking tip. When aligned with the medication such as a pill 9 in the blister pack 8, it allows for a precise striking on the center of pill 9. This makes for quicker and more reliable dispensation of medicine from within the device.

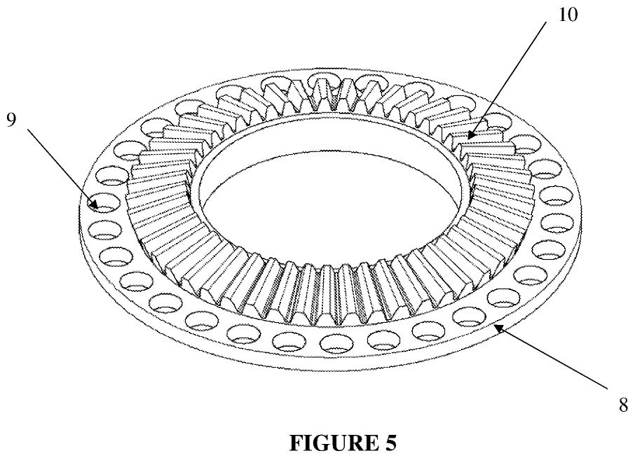

The custom blister pack (FIG. 5) is what houses the medication. Shaped like a pinion, when inserted into the outer shell, its teeth 10 mesh with those 14 of a pinion connected to a stepper motor 3. The stepper motor is engaged when a correct fingerprint is scanned and accepted within the programmed time interval. Since it is 3D printed, the teeth on the blister pack are precise to rotate a predetermined number of degrees in accordance with the number of pills within each pack. The amount of pills contained in the pack are determined by the user's prescription. The blister pack is similarly made of PLA plastic and contains two outer coatings on each side that are made of a thin material, which is easily broken by the striker, yet strong enough to house the pills. This allows the striker to easily punch through and dispense the medication through the bottom hole 12 of the lid 11. The blister pack design also safely contains the medication; it is difficult to remove from or turn inside the device.

Controlled by the use of microcontroller 1 and other components, the electronics combine function with security. The internal system relies on a power supply housed in a simple battery pack 6. These internals rest within the outer shell of the device where they are protected from damage. Once the device is assembled, these internals interlock with the device's bottom lid to rotate the blister pack. The circuit provided gives direct voltage to the mechanisms that are operated in the system. This allows it to be secure and function as intended.

The biometric scanner 1 ensures the security of the device and acts as the first line of defense of the housed drug. Without the correct biometric identifier on the device, the electronics and mechanisms inside will not function. This security mechanism makes sure that the person prescribed the medication is the sole person who can receive it. In one preferred embodiment, a TTL (GT-521F32) fingerprint scanner 1 which is a high intensity DSP chip that processes the fingerprints and analyzes them with extreme precision, holding up to 200 unique IDs, can be used. The scanner 1 is made up of an ultra-thin optical sensor, an ARM Cortex M3 core CPU, and a Universal Asynchronous Receiver/Transmitter (UART) interface. The board's UART interface allows it to be easily mass-produced and scaled. It also interprets a person's finger when it is placed on the scanner 1. The operating voltage of the scanner 1 is from 3.3 volts to 6 volts. The sensor is connected to a microcontroller 4 which is made to support all drivers and hardware of the fingerprint scanner 1 and the rest of the electronics.

In one preferred embodiment functioning with a simple Arduino IDE, the microcontroller is an open source board with Wi-Fi capability. The programming is completed in Arduino with the ability to be implemented into a graphical user interface for pharmacy use. To operate, a compiled library will run a software program allowing the microcontroller to interact with the sensor. The board is compact, holding two 5 volt pin outputs and five 3.3 volt pin outputs. The program can be separated into two parts: enrolling and testing the fingerprint. When arriving at the pharmacy, the unit will undergo the enrolling phase where the patient's fingerprint will be registered in the device. The testing software begins to compile and upon completion, the fingerprint scanner 1 will activate the stepper motor 7 when scanned, allowing the user to take the medication 27.

For the pharmacist to open the locked container, in one preferred embodiment, 4 DC motors may be used to turn to release the device's lid. In one preferred embodiment, Lathan Rectangular Motors which run on 3.3 volts can be used. Once the pharmacist's finger print is recognized, the motors will turn the locking bar and allow the pharmacist to switch the blister pack and enroll a new fingerprint. The GT-521F32 scanner 1 identifies the user's fingerprint in under two seconds with a high accuracy rate. The programming is composed of three main parts: activating the servo motors 5, specifically instructing the fingerprint scanner 1 to perform certain tasks, and maintain control of the internal timer. In one preferred embodiment, a small Nema hybrid stepper motor allows the blister pack to rotate. It moves up to 200 steps per revolution and operates on 5 volts. Once the finger print is accepted, the stepper motor 7 will turn 29, allowing the striker 15 to punch and release the medication 30. Also, in one preferred embodiment, embedded into the device is a circular light ring 2 to alert the user if their pill is ready. The light will flash red if the scanner 1 denies the user (either for the incorrect fingerprint or wrong time interval) or green if it is time for the user's next dose.

The battery 6 supplies power to the board by plugging into the USB connector on the programming board. The stepper motor 7 and the biometric sensor 1 receive power from their five-volt pin connection 21. The stepper motor 7 also connects to digital pin 0 16 and digital pin 1 17. This allows the stepper motor 7 to be programmed with a GND pin 25 to complete the circuit. The biometric sensor 1 is one preferred embodiment utilizes an RX pin 18 and TX pin 19 so that it can receive and store data that is inputted into it. The biometric sensor can also use a GND pin 20 to complete its circuit. One pair of the locking servo motors 5 utilize a 3.3 volt pin connection 22 and a GND pin 23, while the other pair utilizes a separate 3.3 volt pin 26 and GND connection 24. This circuit is run in parallel to power the devices internal electronics.

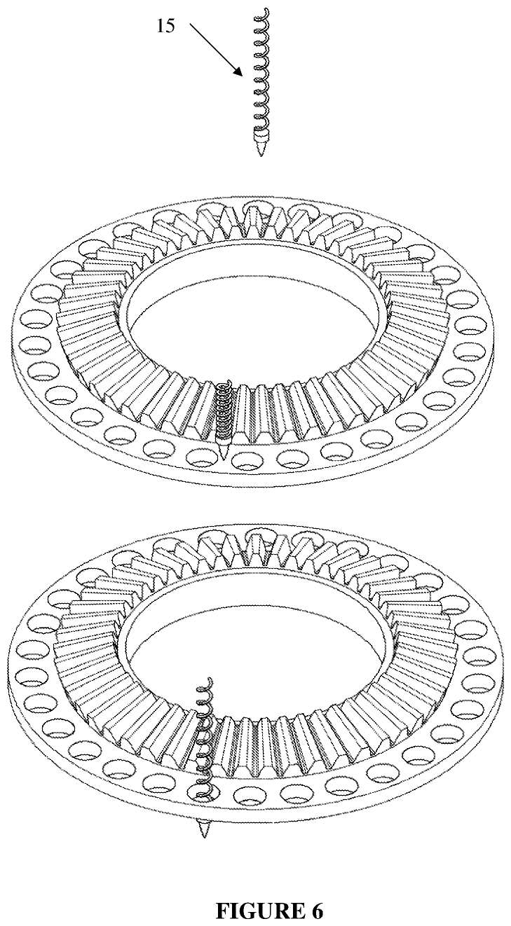

The device is operated through the following steps to ensure the safety of the user. It is first assembled at the pharmacy where the pharmacist inserts the blister pack (FIG. 5) into the bottom lid (FIG. 4) of the device and secures them with the servo motor locking system. The user now must wait until the allotted time has passed before the device will dispense a pill. Indicated by the light ring 2, if the fingerprint is recognized, the internal stepper motor will rotate the blister pack and dispense the pill. It will concurrently disable the scanner 1 from accepting a fingerprint until the proper time has elapsed. The stepper motor is calibrated to rotate the blister pack directly over the hole in the bottom lid. The striker pin (FIG. 6) then pushes out the medication to the user.

One of ordinary skill in the art will appreciate that the systems, apparatus, and methods of the present invention above are exemplary. The present invention can be implemented in various embodiments without deviating from the scope of the invention.

The invention claimed is

1. A device for dispensing medication comprising:
a container configured to store and release individual dosages of medication;
a locking mechanism operatively connected to the container for preventing unauthorized access to the medication stored within the container;
a biometric scanner operatively connected to the locking mechanism and the container configured to unlock the locking mechanism and allow access to the medication in the container only when an authorized person triggers the biometric scanner;
a release mechanism configured to allow a single dosage of medication to be released from the container at a specified time; and
a control system that can be programmed to control operation of the release mechanism to allow the single dosage of medication to be released from the container at a specified time.

2. The device of claim 1 wherein the container comprises:
an outer shell; and
a lid wherein the outer shell and the lid can be connected so as define a secure space in which the medication can be stored such that it cannot be accessed from outside the secure space; and
wherein the locking mechanism is operatively connected to said lid and said outer shell and prevents separation of the lid from the outer shell unless an authorized person triggers the biometric scanner.

3. The device of claim 1 wherein a plurality of individual doses of medication are stored in the container in a plurality of separate packages such that each package contains a single dosage of medicine.

4. The device of claim 1 wherein said biometric scanner is a fingerprint scanner.

5. The device of claim 1 wherein the individual dosages of medication comprise pills.

6. The device of claim 1 wherein said control system comprises a microcontroller.

7. The device of claim 1 further comprising a battery pack operatively connected to said locking mechanism, said biometric scanner, and said control system to provide electrical power thereto.

8. The device of claim 2 wherein the locking mechanism comprises a plurality of servo motors with bars attached thereto mounted on the interior of said outer shell;
wherein each of said plurality of servo motors operate to insert one of said bars into one of a plurality of female notches located on said lid to lock the outer shell and lid together; and
wherein each of said plurality of servo motors operate to remove one of said bars from one of said plurality of female notches located on said lid to unlock the outer shell from the lid.

9. The device of claim 8 wherein the operation of the plurality of servo motors to lock and unlock the outer shell from the lid is initiated in response to an authorized person interacting with the biometric scanner.

10. The device of claim 3 wherein the release mechanism comprises a mechanical striker operated when triggered by said control system to strike one of said plurality of separate packages and create an opening in one of said plurality of separate packages thereby allowing the single dosage of medication to be released from one of said plurality of separate packages and said container through a hole in said lid.

11. The device of claim 3 wherein said plurality of separate packages are operably connected to a circular structure configured to be rotated by a stepper motor located within said outer shell.

12. A device for dispensing medication comprising:
a container configured to store and release individual dosages of medication comprising:
an outer shell comprising a rigid exterior surface defining an interior opening;
a lid comprising a rigid exterior surface with a single hole therethrough; and
a removable blister pack comprising a plurality of packages of individual dosages of medicine that can be mounted within the interior opening of said outer shell;
a fingerprint scanner configured to produce a signal when an authorized person touches said fingerprint scanner;
a locking mechanism operatively connected to the container for preventing unauthorized access to the medication stored within the container comprising:
a plurality of notches on said lid; and
a plurality of servo motors mounted within the interior opening of said outer shell each configured to insert a bar into each one of said plurality of notches on said lid to lock said lid and said outer shell together and remove each of said bars from each of said plurality of notches to unlock said lid from said outer shell in response to said signal from said biometric sensor;
a release mechanism comprising a mechanical striker configured to break open one of said plurality of packages of individual dosages of medicine located proximate to said hole in said lid and allow a single dosage of medication to be released from the container through the hole in said lid after a specified period of time and in response to the signal from said finger print scanner produced when an authorized person contacts said finger print scanner with a finger;
a control system comprising a microcontroller wherein said microcontroller is programmed to control operation of the release mechanism to allow the mechanical striker of said release mechanism to break open one of said plurality of packages and allow a single dosage of medication contained in one of said plurality of packages to be released from the container to a patient at a specified time; and
a stepper motor located with said the interior opening of said outer shell of said container operably configured to rotate said blister pack to position one of said plurality of packages of individual dosages of medicine proximate to said hole in said lid; and
a battery pack located within the interior opening of said outer shell of said container operably connected to said finger print scanner, said locking mechanism, said stepper motor, and said control system configured to provide electrical power thereto.

13. The device of claim 12 wherein the individual dosages of medicine consist of pills.

14. The device of claim 12 wherein the container is constructed of plastic.

15. A method of dispending individual dosages of medicine stored in a blister pack in a secure container comprising:
activating a fingerprint scanner operably connected to said secure container when touched by an authorized user of said medicine after a specified interval of time since the fingerprint scanner was last activated;
reading a fingerprint of said authorized user of said medicine when detected by said fingerprint scanner;
comparing said fingerprint to a directory of fingerprints of authorized users stored within a microcontroller operably connected to said secure container;
engaging a stepper motor comprising a shaft located within said secure container if said fingerprint matches the fingerprint of an authorized user;
turning a pinion gear connected to said shaft of said stepper motor and operably connected to said blister pack to advance said blister pack to position whereby an individual dosage of medicine is proximate to a mechanical striker;
striking the blister pack with the mechanical striker to break the blister pack containing the individual dosage of medicine proximate to the mechanical striker; and
dispensing the individual dosage of medicine from the blister black to the authorized user.

16. The method of claim 15 wherein said individual dosage of medicine comprises a pill.

17. The method of claim 16 wherein said secure container comprises an outer shell and a lid.

18. The method of claim 16 wherein said blister back was placed in said outer shell and said outer shell and said lid were locked together to form said secure container.
